(19) United States
(12) Patent Application Publication
(10) Pub. No.: US 2015/0104400 A1
(43) Pub. Date: Apr. 16, 2015

(54) TOPICAL DELIVERY AND ADMINISTRATION SYSTEM FOR STABILIZED PROTECTION AGENT, COMPOSITIONS AND METHODS OF MAKING SAME

(71) Applicant: PANKAJ MODI, Ancaster (CA)

(72) Inventor: PANKAJ MODI, Ancaster (CA)

(21) Appl. No.: 14/052,183

(22) Filed: Oct. 11, 2013

(51) Int. Cl. A61K 8/97; A61K 8/64; A61Q 17/04; A61K 8/02; A61Q 19/08; A61K 8/65; A61K 8/55

(52) U.S. Cl. CPC A61K 8/97; A61K 8/65; A61K 8/64; A61K 8/553; A61K 8/0291; A61Q 19/08; A61Q 17/04; A61K 2800/52
USPC 424/60; 424/59

(57) ABSTRACT

The present invention provides a method and product for topical delivery and administration of stabilized protection agents and compositions thereof and methods for making the composition and use thereof. The method and system comprises pharmaceutical compositions for facilitating topical delivery of protection agents and skin anti-photoaging agents for inhibiting cellular degradation in response to sun damage. The pharmaceutical compositions are topically applied on a patient and are generally in a cream, lotion or gel form.

### FIELD OF THE INVENTION

The present invention relates to the field of anti-photoaging and formulations for use in conjunction with a method and product for topical delivery and administration of stabilized protection agents and compositions thereof and methods for making the composition and use thereof. The method and system comprises pharmaceutical compositions for facilitating topical delivery of protection agents and skin anti-photoaging agents for inhibiting cellular degradation in response to sun damage. The compositions are topically applied on a patient in a cream, lotion or gel form and are in a dissolved and suspended micellar-like form for use as a component in a topical system for delivery of the inhibitor system. The compositions are particularly effective in topical applications. The present invention further relates to methods for preparing and using these compositions, as well as methods for enhancing the stability and the topical retention on the skin of the user of the cellular degradation inhibiting agent.

### BACKGROUND OF THE INVENTION

For storage stability and convenience of handling of certain protective agent compositions are often formulated as a lyophilized or vacuum dried powder or dried extract. The dry powder is typically reconstituted with a suitable fluid. However, such powders are often difficult to solubilize and, upon application, result in a residue (often whitish in nature and chalky) when applied to a person's skin. Furthermore, they may tend not to be retained on the skin. Finally, many compounds are difficult to stabilize and there may be a loss of compound or activity during the formulation or reconstitution and any period or storage. Stability problems can occur as a result of inability to solubilize, degradation, dimerization, and/or polymerization. Various excipients have been used with differing degrees of success to try and stabilize an active agent in a pharmaceutical composition. Stabilizers may also be effective in reducing adhesion of the active agent to surfaces, such as the surfaces of laboratory glassware, vessels, the vial in which the pharmaceutical composition is reconstituted or the inside surface of a syringe used to inject the pharmaceutical composition. As well as being able to stabilize an active agent in a composition, an ideal stabilizing agent should have negligible immunogenicity when administered to a human patient.

It has proven particularly difficult to stabilize active agents for cosmetic or topical dermatological preparation. Various types of active protective agents are useful for the prophylaxis and treatment of cosmetic and dermatological skin changes, such as skin aging and, in particular, aging induced by oxidative or degenerative processes. It is also desirable to prepare preparations that can be administered topically to enhance the inhibiting effect on cellular degeneration caused by the various effects of UVA and UVB radiation from the sun.

The dangers of free radical production by ultraviolet (UV) light exposure have become an enormous public health issue, worsened by the deleterious effects of greenhouse gases on the ozone layer and global warming. In 2000, the NIH listed solar radiation as a known human carcinogen for the first time.

UV radiation is divided into three categories according to wavelength: UVA rays range from .about.320-400 nm (nanometers), UVB rays from .about.290-320 nm, and UVC rays from .about.100-290 nm. Both UVA and UVB are found in sunlight, while UVC is completely filtered by the ozone layer. UVA is .about.100 times more prevalent than UVB; and because UVA has the longest wavelengths, it penetrates the skin more deeply than UVB. UVA is subdivided into two wavelength ranges which are known as UVA-I (.about.340-400 nm) and UVA-II (.about.320-340 nm). While all UV light directly interacts with the skin's DNA to induce mutations, the primary method of destruction is the formation of reactive oxygen species (ROS), destructive molecules known as a “free radicals”, which cause cumulative damage to healthy cells, including cells of the immune system.

Non-melanoma skin cancer is by, far the most common cancer seen in man. Melanoma, the most deadly form of skin cancer, has recently been identified as the cancer with the sharpest increase in prevalence among all cancers. UV light is a primary carcinogen in non-melanoma skin cancers, and a co-carcinogen in melanomas. Most of the more than 1 million eases of non-melanoma skin cancer diagnosed in the United States each year are sun-related. With ozone depletion progressing and recreational time in the sun increasing, the rates of skin cancer are expected to increase in the future, despite already being at epidemic levels in certain countries such as Australia. Accordingly, the development of improved sunscreens and sunblocks is critical to protect against the deleterious health effects of UV light.

Photoaging is a term presently used to describe the changes in appearance and/or function of human skin as a result of repeated exposure to sunlight, and especially regarding wrinkles and other changes in the appearance of the skin.

Solar radiation reaching the earth's surface that effects and enables various animals, including humans, comprises ultraviolet (UV) (.lambda.<400 nm), visible (400 nm<.lambda.<700 nm), and infrared (IR) (.lambda.>700 nm). UV radiation is generally divided into UVA (320-400 nm), UVB (290-320 nm), and UVC (<290 nm); UVC radiation is blocked from reaching the earth's surface by stratospheric ozone. The ultraviolet (UV) component of sunlight, particularly UVB, is generally believed to be the principal causative agent in photoaging.

The extent of UV exposure required to cause photoaging is not currently known, although the amount required to cause erythema (reddening, commonly seen as sunburn) in human skin is known and quantified empirically as the “minimal erythemal dose” (“MED”) from a given UV source. UVB wavelengths of 290-300 nm are the most erythmogenic. The effectiveness of UV radiation in causing erythema decreases rapidly as the UV wavelength is increased beyond about 300 nm; wavelengths of 320 nm and 340 nm are, respectively, one hundred and one thousand times less potent at causing skin reddening than wavelengths of about 298 nm. Repeated exposure to sunlight at levels that cause erythema and tanning are, nevertheless, commonly associated with photoaging.

Erythema from UVB is suggested to be a function of the total radiation exposure, not the intensity of the radiation exposure. According to Physiology, Biochemistry, and Molecular Biology of the Skin, 2nd Ed., ed. by L. A. Goldsmith (New York: Oxford Univ. Press, 1991), UVA is considered both melanogenic and erythemogenic and UVA exposure induces synthesis of a 32 kDa stress protein in human skin, as well as immediate erythema not apparent after UVB exposure.

Photoaging in human skin is characterized clinically by coarseness, wrinkles, mottled pigmentation, sallowness, laxity, eventually premalignant, and ultimately malignant neoplasms. Photoaging commonly occurs in skin that is habitually exposed to sunlight, such as the face, ears, bald areas of the scalp, neck, forearms, and hands.

Sunscreens are commonly used to prevent photoaging of skin areas that are exposed to sunlight. Sunscreens are topical preparations that contain ingredients that absorb, reflect, and/or scatter UV light. Some sunscreens are based on opaque particulate materials, among them zinc oxide, titanium oxide, clays, and ferric chloride. Because such preparations are visible and occlusive, many people consider these opaque formulations cosmetically unacceptable. Other sunscreens contain chemicals such a p-aminobenzoic acid (PABA), oxybenzone, dioxybenzone, ethylhexyl-methoxy cinnamate, octocrylene, octyl methoxycinnamate, and butylmethoxydibenzoylmethane that are transparent or translucent on the skin. While these types sunscreens may be more acceptable cosmetically, they are still relatively short-lived and susceptible to being removed by washing or perspiration. Sunscreen formulations for use on human skin are well-known and many different types are commercially available to satisfy diverse consumer needs.

For example, sunscreen formulations having different sun protection factor (SPF) values are available, thus allowing consumers to choose the amount of protection desired. SPF values range from zero upward with higher values indicating greater amounts of sun protection. SPF values of 2-4 indicate minimal sun protection, 4-6 indicate moderate sun protection, 8-15 indicate maximal sun protection and above 15 indicate ultra sun protection.

Not only SPF values but aesthetics must be considered in developing consumer acceptable sunscreen compositions. Higher SPF formulas require, of course, higher levels of sunscreen. With inorganic sunscreens higher levels unfortunately leave a visible residue, sometimes referred to as “whitening” on the skin. Whitening detracts from a product's aesthetics. Consumers desire their cosmetics to be unobtrusive, i.e. invisible. Inorganic sunscreens such as titanium dioxide and zinc oxide are particularly prone to the whitening effect. U.S. Pat. No. 5,028,417 (Bhat et al) sought to overcome the whitening problem through use of an ultrafine titanium dioxide of particle size less than 10 nm. EP 0 433 086 A1 (Cole et al) describes a combination of titanium dioxide and zinc oxide in relative particle sizes of less than 35 nm and 50 nm, respectively.

As noted above, the generally accepted etiology of photodamage to skin involves an exposure to sunlight sufficient to cause erythema (sunburn or reddening; literally a flush upon the skin), and it is now known that sufficient UVB radiation does cause erythema. This philosophy dictates that present compositions and methods for inhibiting photoaging include the use compounds that block or absorb UVB, and that such compositions need be used only when there is sufficient likelihood that exposure to sunlight will result in erythema. More recent sunscreen compositions include combinations of compounds that block both UVA and UVB radiation.

It has been suggested that UV solar radiation induces reactive oxygen species (ROS) in the skin. Rieger, M. M. Cosmetics and Toiletries (1993) 108:43-56 reviews the topical application of known antioxidants to the skin for reducing the presence of ROS.

Retinoids have been used as therapy to improve the appearance of sundamaged skin. U.S. Pat. No. 4,877,805 describes the treatment of photoaged skin. The patent indicates that there is little point in beginning the application of a retinoid to treat photodamage until the effects of aging begin to appear. Several studies have investigated improving the appearance of existing photodamaged skin with the use of all-trans retinoic acid. G. D. Weinstein et al., “Topical Trentinoin for Treatment of Photodamaged Skin,” Arch. Dermatol., 127:659-665 (May 1991); J. S. Weiss et al., “Topical Tretinoin Improves Photoaged Skin,” J. Amer. Med. Assn., 259(4):527-532 (Jan. 22/29, 1988).

Matrix metalloproteinases (MMPs) are a family of enzymes that play a major role in physiological and pathological destruction of connective tissue, especially collagen. Various types of collagen and collagenases (types of MMPs) are known in this field. Inhibitors of MMPs (i.e., direct inhibitors of the proteinase) and of molecular pathways (i.e., inhibitors of AP-1) that affect MMP expression are known in other fields.

In summary, the state of the art considers that photodamage is caused primarily by UVB radiation, and that presently available sunscreens are sufficient to prevent photodamage. “Dr. Ceilley [former President of the American Academy of Dermatology] believes that staying out of the sun and using sunscreen could have prevented many of the skin cancers that he treats in his practice, as well as the premature wrinkles that his patients are concerned about.” Skin SAVVY, Amer. Acad. Dermat. supp. to USA Today, May 1997.

However, it has been recently shown that in circumstances where there is UV exposure insufficient to cause erythema, there may still be connective tissue damage at the dermal level via MMP induction. Thus, suberythemal doses of UV radiation induce MMPs that degrade skin connective tissue and thus are likely responsible for photoaging. A UV exposure (with UVA and/or UVB) insufficient to cause erythema nevertheless is sufficient to cause photodamage via MMP induction. As such, the term “photodamage” should be redefined in the art so as not to require erythema. Thus, a combination of UVA and/or UVB radiation can significantly damage the skin. For purposes of the present invention, it broadly includes preventing photodamage from UVA and/or UVB radiation, especially before clinical signs of photodamage are presented.

The process that leads to skin aging and wrinkles is complex. A primary cause of wrinkling is a build-up of free radical toxic plaque that binds to collagen and elastin fibers, causing the skin's supportive structure to become inflexible and unhealthy. Laugh lines, smile lines, crow's feet or facial creases appear in areas where repeated muscle movement occurs. Thus, it would be desirable to be able to deliver free radical scavengers to the skin. Thus, there was a need for newer methods for stabilization of active agents for the preparation of topical and cosmetic preparation. There was also a need for improved systems for delivery of active agents to the skin. The present invention addresses those needs and provides a delivery system to permit the application and retention of anti-photoaging compositions to reduce damage and cellular degradation from exposure to both UVA and UVB radiation, retard photoaging, provide an immunomodulatory effect and otherwise assist in MMP (matrix metalloproteinase) inhibition.

Certain oral supplements have been reported to provide protection from sunburn. For example, astaxanthin, a carotenoid pigment, has been reportedly used for sun protection, see, e.g., U.S. Pat. No. 6,433,025; the mechanism of action is believed to be at least in part due to the antioxidant properties of astaxanthin. Extracts of ferns of the genus Polypodium have been reported to be photoprotective, as discussed in U.S. Pat. No. 5,614,197, possibly due to an immunomodulatory effect. The use of antioxidants and other nutritional substances to retard photoaging and its mechanisms (primarily through MMP (matrix metalloproteinase) inhibition for sun protection are discussed in U.S. Patent Application 20050058709.

In the fight against cancer, certain studies have demonstrated that polypodium with green tea has beneficial effects. Most of the studies relate to antimutagenic effects upon ingestion of this substance. There has, however, been one proposal for topically applying polypodium with green tea on skin as protection against ultraviolet radiation B-induced photocarcinogenesis in murine skin. See “Proposal to the American Institute for Cancer Research”, (Dec. 22, 1989) by Hasan Mukhtar and Cancer Letters, 42 (1988) pages 7-12, by Mukhtar et al.

Although the exposure of individuals to moderate sunlight has many beneficial effects, including the synthesis of vitamin D and the killing of certain pathogens, over-exposure of human skin to sunlight and, in particular, to the ultraviolet band of the spectrum, has many deleterious effects, including sunburn, phototoxicity, photoallergic reactions, photoaging, and the promotion of skin cancers. As a result of concerns about the deleterious effects of over-exposure to sunlight, much research has been directed to the development of both topical and systemic photoprotective agents and preparations for use in cosmetics and sunscreens (for reviews, see M. A. Pathak, Dermatologic Clinics 4(2):321-334 (1986); M. A. Pathak, “Topical and Systemic Photoprotection of Human Skin Against Solar Radiation,” in H. W. Lim and N. A. Sotek, (eds.), Clinical Photomedicine, New York, Marcel Dekker, Inc. (1993); M. A. Pathak and T. B. Fitzpatrick, “Preventive Treatment of Sunburn, Dermatoheliosis, and Skin Cancer with Sun-Protective Agents,” in Fitzpatrick, T. B., et al., (eds.), Dermatology in General Medicine, 4th Edition, New York, McGraw-Hill, (1994)).

Extracts of Polypodium leucotomos have been found effective in the treatment of psoriasis, atopic dermatitis and other skin disorders (see, e.g., H. Corrales Padilla, et al., Int. J. Dermatol. 13(5):276-282 (1974); D. Jimenez, et al., Allergol. et Immunopathol. 15(4):185-189 (1987)). In these settings, the extract was found to cause decreases in hyperkeratosis, parakeratosis, epidermal mitosis, epidermal thickening, epidermal prolongations, and the severity and extent of epidermal lesions.

### SUMMARY OF THE INVENTION

The present invention addresses the need for improved dermatological delivery of active protective agents by stabilizing those active agents and pemitting them to be dissolved and suspended in a micille-like formation and for enhancing the retention of the active ingredients on the skin. The invention also provides compositions prepared using the method for the topical delivery of at least one active protective agent.

In one aspect of the invention, the method comprises suspending an active protective agent in a phospholipid micelle-like coating. The active agent is then admixed with a solution containing collagen and/or elastin.

In another aspect of the invention, a composition for topical delivery of at least one active protective agent is provided. The composition comprises the active protective agent in a micelle-like suspension and an acceptable carrier. The composition also optionally includes elastin, preferably low molecular weight cross-linked elastin.

Polypodium extract comprises an extract of a plant selected from the group consisting of Polypodium aureum, Polypodium crassifolium, Polypodium decumanum, Polypodium lanceolatum, Polypodium leucotomos, Polypodium percussum, Polypodium triseriale and Polypodium vulgare. In another aspect of the invention, the extract is more preferably Polypodium leucotomos extract.

In one preferred embodiment, the active protective agent is selected from the group consisting of Polypodium leucotomos, Vitamin A, Vitamin C, Vitamin E, Zinc, Selenium, lycopene, N-Acetyl-Cysteine, natural plant extracts of Grape Skin, Bilberry and Green Tea and combinations thereof.

Another mode of the invention provides a composition prepared by combining at least one carotenoid with a Polypodium extract in connection with the micelle-like suspension delivery system where the preferred mode employs an extract of Polypodium leucotomos as the Polypodium extract.

In another aspect, the invention provides a pharmaceutical composition including an effective photoprotectant amount of at least one carotenoid and a Polypodium extract, together with a pharmaceutically-acceptable carrier.

In preferred embodiments of the pharmaceutical composition, the at least one carotenoid is a carotenoid selected from the group consisting of astaxanthin, lycopene, canthaxanthin, .beta.-carotene, lutein, and zeaxanthin, more preferably astaxanthin and/or lycopene. In preferred embodiments of the pharmaceutical composition, the at least one carotenoid is a carotenoid selected from the group consisting of astaxanthin, lycopene, canthaxanthin, .beta.-carotene, lutein, and zeaxanthin, more preferably astaxanthin and/or lycopene.

In one of the preferred embodiments, retinoids are used to assist in the prevention of photodamage in conjunction with one or more of the active protective agents. Compounds which may also be useful in preventing photodamage by inhibiting the production and/or activity of MMPs are termed “antioxidants” and may prevent erythema and are also employed in the embodiment to reduce the concentration of MMPs in UV-exposed human skin and where the use does not correlate with inhibiting UV-mediated increases in MMPs.

Preferred phospholipids include sphingosine and cerebroside. In a preferred embodiment, the micelle comprises both sphingosine and cerebroside. In one particular embodiment, the sphingosine and cerebroside are combined in equal amounts.

In a further preferred embodiment, the composition is formulated in a format selected from the group consisting of a cream, a lotion, a spray, an ointment, a gel, a powdered mask, a paste, a cleanser, and a foundation.

The composition may optionally include an additive selected from the group consisting of a perfume, colorant, thickening agent, vegetable oil, emulsifier, solvent, pH adjusting agent, antiseptic agent, preservative, vitamin, sun-block, surfactants and combinations thereof. Various physical sunscreen agents such as titanium dioxide, silicone-treated titanium dioxide, zinc oxide, ferrous oxide, ferric chloride, talc, chromium oxide, or cobalt oxides may be included. Alternatively or in addition, a chemical sunscreen agent such as para-amino benzoic acid, esters of para-amino benzoic acid, salicylates, cinnamates, benzophenones, dihydroxyacetone, parsol 1789, or melanin may be included. The preparations may contain at least 1%, 10%, 25%, or 50% Polypodium extract by weight. In addition, the preparations may provide a sun protection factor (SPF) for the minimal erythematic dose (MED) evaluated at 24 hours of at least 2, 5, 10 or 15 when applied at 2 μl/cm2 to normal skin of Types I to Type IV.

In a preferred embodiment, the present invention provides a composition comprising:

approximately 1 to 40% w/w collagen;
approximately 1 to 40% w/w elastin;
approximately 0.1 to 15% sphingoside phospholipid;
approximately 0.1 to 15% cerebroside phospholipid;
a carotoid approximately 5% by weigh and preferrabley between at least 0.5%-25%; and,
at least 1%, 10%, 25%, or 50% Polypodium extract by weight..

In another aspect, the present invention provides a method for making a stabilized active protective agent composition. The method comprises the steps of:

i. preparing a phospholipid solution comprising a phospholipid in a solvent;
ii. admixing the phospholipid solution with an active protective agent so as provide a phospholipid micelle-like dissolved and suspended active protective agent solution;
iii. combining the carotenoid and maintaining the activee protective agent in a dissolved, micella-like suspension; and,
iv. combining the suspension with a solution of solubilized collagen and elastin.

In a preferred embodiment of the method, the phospholipid is selected from the group consisting of sphingosine, cerebroside and combinations thereof. More preferably, sphingosine and cerebroside are used in equal amounts.

In a further preferred embodiment, the solvent used is an alcohol. Preferred alcohols include isopropanol, ethanol and mixtures thereof.

In another preferred embodiment, the solution of solubilized collagen and elastin comprises equal amounts of collagen and elastin.

Active agents that can be used in the method of the invention include botulinum toxin, hyaluronic acid, Vitamin A, Vitamin C, Vitamin E, Zinc, Selenium, lycopene, N-Acetyl-Cysteine, natural plant extracts of Grape Skin, Bilberry and Green Tea, vasodilators, hormones, growth factors, vaccine agents, drugs, therapeutic proteins, small molecules, antiperspirant agents, analgesics and combinations thereof. Preferred active agents for use in the method are hyaluronic acid and/or botulinum toxin.

The method of the present invention may include the further step of adding a permeation enhancing compound. The enhancing compound is preferably selected from the group consisting of d-limonene, allantoin, fulvic acid, myrrh, hydroquinone glyquin, quillaja saponaria, and acanthophyllum squarrusom.

In a preferred method of the invention, about 0.1 to 15 wt/wt % of sphingosine and about 0.1 to 15 wt/wt % of cerebroside are used to form the micellar coating.

In a further preferred method, additional ingredients are added to form a cream, lotion, gel, ointment, or spray.

In a further aspect of the invention, a method of treating skin is provided. The method comprises applying the composition described above daily to areas of the skin which are wrinkled or damaged and preferrably prior to exposure to sunlight.

The present invention addresses many of the problems of the prior art. The compositions can be formulated as a cream or lotion and can be stored at room temperature for extended periods of time without any loss of activity of the active ingredient. It can also be applied without having a whitening appearance and may provide extended retention on the skin's surface to provide extended protection.

Certain compositions of the invention are useful to reduce fine lines and wrinkles, increase the moisture level of the skin, increase skin elasticity and resilience, increase the firmness of the skin, improve skin tone, texture and overall radiance, diminish bags under the eyes, rejuvenate the skin, prevent damage from chemical stress, protect the skin from UV rays and free-radical damage, promote wound healing and remove irregular pigmentation.

### BRIEF DESCRIPTION OF THE DRAWINGS

These and other features of the invention will become more apparent from the following description in which reference is made to the appended drawings wherein:

FIG. 1 illustrates a chart detailing a testing protocol for the MED testing for NB-UVB light;

FIG. 2 is a photograph of the test area of Test Subject No. 1 with demarcations for test window identified thereon in relationship to a 10% polypodium MED test employing the composition of the present invention;

FIG. 3 is a series of photographs of Test Subject No. 1 with various levels of Polypodium and test protocol employing the composition of the present invention;

FIG. 4 is a photograph of the test area of Test Subject No. 2 with demarcations for test window identified thereon in relationship to a 10% polypodium MED test employing the composition of the present invention;

FIG. 5 is a series of photographs of Test Subject No. 2 with various levels of Polypodium and test protocol employing the composition of the present invention;

FIG. 6 is a photograph of the test area of Test Subject No. 3 with demarcations for test window identified thereon in relationship to a 10% polypodium MED test employing the composition of the present invention;

FIG. 7 is a series of photographs of Test Subject No. 3 with various levels of Polypodium and test protocol employing the composition of the present invention.

### DETAILED DESCRIPTION OF A PREFERRED EMBODIMENT OF THE INVENTION

The present invention provides methods of preparing a stabilized composition for enhanced topical delivery of at least one active protective agent and compositions prepared by the methods.

The method of the invention comprises dissolving an active protective agent in a phospholipid micelle-like suspension. The micelle-like suspension is then preferably combined with a base composition that includes collagen. Briefly, the phospholipid is dissolved in a suitable solvent, such as an alcohol. For example, the phospholipid may be dissolved in ethanol or a mixture of ethanol and isopropanol. The alcohol is removed by, for example, rotary vacuum evaporation. The active protective agent is then dissolved and added to permit it to form a micelle-like suspension. The active protective agent thus becomes encapsulated by a phospholipid micelle-like suspension structure. This suspension can then be combined with a base solution comprising collagen. It has also been found to be advantageous to include elastin, especially cross-linked low molecular weight elastin, in the base composition.

Collagen can act as a stabilizer. In addition, collagen helps improve the tissue's underlying foundation and contributes to hydration. Collagen is able to penetrate the skin without the aid of any penetration enhancers to assist in the regenerative process.

While various types of phospholipids can be used to form the micelles, a sphingoside is one preferred phospholipid. Sphingolipid or sphingosine-1-phosphate has been recognized as a bioactive molecule involved in the regulation of cell growth, differentiation, survival, and chemotaxis as well as angiogenesis and embryogenesis. Other species of ceramides or sphingolipids such as (N-acyl-sphingosine) and dihydroceramide (N-acyl sphinganine) are also useful in the present invention. Galactosylceramide (cerebroside), a metabolite of sphingolipids is also a preferred phospholipid. Cerebroside is a myelin related protein that plays an important role in the regulation of cell growth, differentiation, survival, and chemotaxis. Cerebroside sulfates are important membrane constituents.

Both sphingosine and cerebroside have been found to be excellent phospholipids for use in the method of the present invention. While either of these phospholipids can be used alone, combinations of sphingosine and cerebroside are particularly effective. When used in equal amounts, sphingosine and cerebroside form a micelle structure that provides a very effective vehicle for delivery of active protective agents and for surface retention on the skin of the indiviudal of the protective agent. Other phospholipids, such as phosphatidyl choline or phosphatidyl serine are also highly effective, either alone or in combination. Other phospholipids that can form a micellar-like structure and suspension are also useful in the invention.

A preferred method for preparing a stabilized composition for topical application is described in Example 1 below. The method of the present invention was used to stabilize in a cream/lotion format suitable for application to the skin. Briefly, equal amounts of collagen and elastin are solubilized in saline. In a separate flask, equal amounts of sphingosine and cerebroside are dissolved in alcohol. The alcohol is then removed. The Polypodium is dissolved and is then added to the flask and the flask is swirled to coat the Polypodium with a phospholipid micelle-like coating resulting in a suspension. This suspension is then added to the solution of collagen and elastin.

The pharmaceutical compositions of the present invention comprise a therapeutically effective amount of (i) at least one carotenoid and (ii) a Polypodium extract, formulated together with one or more pharmaceutically acceptable carriers. As used herein, the term “pharmaceutically acceptable carrier” means a non-toxic, inert solid, semi-solid or liquid filler, diluent, encapsulating material or formulation auxiliary of any type.

Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions of this invention can be administered to humans and other animals topically.

In certain embodiments, the invention provides topical formulations. In addition to the at least one carotenoid and the Polypodium extract, a topical preparation may include physical sunscreen agents, chemical sunscreen agents and/or cosmetic agents. In particular, a physical sunscreen agent such as titanium dioxide, silicone-treated titanium dioxide, zinc oxide, ferrous oxide, ferric chloride, talc, chromium oxide, or cobalt oxides may be included. Alternatively or in addition, a chemical sunscreen agent such as para-amino benzoic acid, esters of para-amino benzoic acid, salicylates, cinnamates, benzophenones, dihydroxyacetone, parsol 1789, or melanin may be included.

The term “carotenoid,” as used herein, is art-recognized and refers to a member of a family of organic pigments characterized by a polyene chain. Examples of carotenoids include carotenes, such as lycopene, .alpha.-carotene and .beta.-carotene, and xanthophylls, such as canthaxanthin, astaxanthin, lutein and zeaxanthin.

The term “Polypodium extract” extract, as used herein, refers to an extract of a plant of the genus Polypodium. Polypodium species include Polypodium aureum, Polypodium crassifolium, Polypodium decumanum, Polypodium lanceolatum, Polypodium leucotomos, Polypodium percussum, Polypodium triseriale and Polypodium vulgare. An extract of Polypodium for use in the compositions and methods of the present invention should have photoprotectant activity. Plant extracts can be described based on a reference compound found in the plant, or can refer to a concentration (e.g., weight/weight) of the total extract compared to the whole or dried plant material. An extract can be, e.g., a 30:1 or 50:1 extract.

The terms “photoprotective” and “photoprotectant”, as used herein, refer to the property of protecting skin from damage due to exposure of the skin to ultraviolet (UV) radiation (e.g., sunlight), including UVA (long-wave UV), UVB (shorter-wave UV), or both UVA and UVB, or to a composition having such a property. Compounds and compositions of the invention are generally capable of achieving one or more of the following effects: the inhibition or retardation of skin inflammation, erythema or sunburn reaction and tissue damage to skin and/or the inhibition or retardation of the immediate pigment darkening reaction and/or the inhibition or retardation of the delayed tanning reaction and/or the inhibition or retardation of phototoxic reaction produced by psoralens. A compound or composition providing such photoprotection is said to be “photoprotective” and may be referred to as a “photoprotectant.”

While the description herein generally refers to sunburn or sun damage, it will be appreciated that the invention also related to compositions, kits and methods for preventing, treating, or ameliorating damage to skin caused by exposure to ultraviolet radiation from sources other then the sun (e.g., from tanning beds and the like). In order to repair oxidative stresses and mop up free radicals, a variety of vitamins, cofactors, minerals, antioxidants, quenchers or the like may be included.

Some examples of suitable topical vitamins include, but are not limited to: vitamin A as esters (such as retinyl palmitate, retinyl acetate), as alcohol (such as retinol), as acid (such as retinoic acid), as its natural plant form beta-carotene (retinaldehyde dimer); vitamin C as acid (ascorbic acid), as a salt (such as sodium or magnesium ascorbyl phosphate), as a fat (such as ascorbyl tetraisopalmitate), or as an ester-C; vitamin E as a fat (such as alpha tocopherol), or other; and the like.

An example of suitable topical cofactors includes, but is not limited to: coenzyme Q10 (CoQ 10), and the like.

Examples of suitable topical minerals include, but are not limited to: zinc, magnesium, silicone and the like.

Some examples of suitable topical antioxidants and/or quenchers (which may also have protective characteristics) include, but are not limited to, alpha-lipoic acid, lipochromalin-6, idebenone, coffee berry, green tea polyphenols, Polypodium leucotomos (a fern plant extract), Tinogard TS™ (octadecyl di-t-butyl-4-hydroxyhydrocinnamate), Tinogard NOA™ (tetrabutyl ethylidinebisphenol), Tinogard Q™ (tris(tetramethylhydroxypiperidinol)citrate, an excited state quencher (ESQ) of molecules, or their excited-state intermediaries, such as UV blockers, e.g. avobenzone or the like), manuka oil (from Leptospermum scoparium (manuka) honey), enzogenol (from Pinus radiata pine bark), and the like. Both of the latter 2 originate in New Zealand and have potent antioxidant and wound healing capabilities.

Optimally, the ingredients in this class compliment all the requirements of, and other ingredients in, the formulation; and also remain optimally active on the cutaneous/adherent aspect of the user's skin.

Accordingly, an embodiment may contain vitamins, cofactors, minerals, or antioxidants and/or quenchers in concentrations—either solely, or in combination with each other—of about 0.1% to about 10% by weight (including about 0.5%, 1%, 2%, 3%, 4%, 5%, 6%, 7%, 8%, and about 9%, also including percentages between these recited percentages, as well as ranges of percentages bordered on each end by the recited percentages).

The topical composition of the present invention further comprises a natural extract component. The natural extract component may comprise an extract derived from a plant, fruit, fungus, or a mixture thereof. The amount of a natural extract component may be adjusted such that a final cosmetic product comprises from about 1.0% to about 25% by weight of natural extract component. The natural extract component preferably has high antioxidative capacity, MMP inhibitory activity, and SOD inducing ability, thereby providing both short-term and long-term anti-aging benefits to the skin. The short-term anti-aging effects are caused by agents that are antioxidants and/or MMP inhibitors.

Antioxidative agents often contain effective reducing agents such as polyphenols, glutathione, vitamin C, and vitamin E. The long-term anti-oxidative benefits are provided by certain agents that are capable of either stimulating the production of body's endogenous antioxidants (e.g. superoxide dismutase) or enhancing the activity of such endogenous antioxidants. The natural extract(s) used in the present invention may be obtained through conventional methods known in the art, such as solvent extraction, distillation, enfleurage, and ram press method.

Preferably, the natural extract component may comprise the antioxidant Polypodium leucotomos (a fern plant extract from central America), which has been shown in both oral (Fernblock®) and topical forms to be photoprotective against UVB and PUVA-induced phototoxicity (PUVA=Psoralen+UVA treatment for eczema); as well as to be antioxidant and anti-inflammatory; with a dose dependent SPF of 25-90 (topical).

Accordingly, an embodiment may contain this plant extract in a concentration of about 0.1% to about 10% by weight (including about 0.5%, 1%, 2%, 3%, 4%, 5%, 6%, 7%, 8%, and about 9%, also including percentages between these recited percentages, as well as ranges of percentages bordered on each end by the recited percentages).

The compositions and formulations of the invention are useful to reduce the signs of aging by minimizing the photoaging process and assisting in the regenerative process by reducing the free radicals and acting as radical scavengers.

In another aspect of the invention, a pharmaceutical or cosmetic formulation is provided containing an effective amount of an active protective agent and an effective amount of the stabilizing enhancing composition, and a pharmaceutically acceptable carrier suitable for topical administration. The formulation may be in any form suitable for application to the skin. For example, it may take the form of a cream, a lotion, a gel, an ointment, a paste, or a solution. The formulation may include lipozone, micelles, or microspheres. The formulation may be a cosmetic composition that includes in addition to the stabilizers and the active ingredients water and other additives that are normally used in cosmetics. For example, it may include thickening agents, preservatives, emulsifiers, perfumes, dyes or coloring, vegetable or mineral oil, antiseptic agents, acidifying or alkalizing agents, vitamins, anti-UV agents, surfactant, solvents, pH stabilizing agents, and other active ingredients known to be effective on the skin. The cosmetic composition may also be provided as skin foundation, lip balm, etc.

Liquid dosage forms for topical administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Other skin enhancing active protective agents which can delivered via the methods and compositions of the present invention include various types of anti-oxidants. Some examples of free radical scavengers that maybe included in compositions of the invention include, but are not limited to, Vitamins A, C and E, the minerals Zinc and Selenium, lycopene, the amino acid N-Acetyl-Cysteine and natural plant extracts of Grape Skin and Bilberry.

In another aspect of the invention, the delivery system of the present invention can be used to deliver agents that promote healing. For example, Polypodium and other protective agents can be suspended in a phospholipid micelle-like and then combined with collagen and/or elastin in a lotion or cream formulation and applied to the skin. Without being limited by the explanation, it is thought that the formulation of the protection agents in a topical composition according to the invention enhances the protection and the rate of scavenging for free radicals and otherwise damaged skin.

Liquid dosage forms for topical administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Examples of further active agents that can be delivered in compositions of the invention include, but are not limited to In certain embodiments, the invention provides topical formulations. In addition to the at least one carotenoid and the Polypodium extract, a topical preparation may include physical sunscreen agents, chemical sunscreen agents and/or cosmetic agents. In particular, a physical sunscreen agent such as titanium dioxide, silicone-treated titanium dioxide, zinc oxide, ferrous oxide, ferric chloride, talc, chromium oxide, or cobalt oxides may be included. Alternatively or in addition, a chemical sunscreen agent such as para-amino benzoic acid, esters of para-amino benzoic acid, salicylates, cinnamates, benzophenones, dihydroxyacetone, parsol 1789, or melanin may be included.

The compositions of the present invention may contain a single active agent or multiple active agents in the same composition. For example, a composition for topical delivery may comprise micelle encapsulated or suspended protective agents of retenoid and/or carotenoids or both. Various combinations of active agents are contemplated for inclusion in the compositions of the invention.

The present invention has been described with regard to one or more embodiments. However, it will be apparent to persons skilled in the art that a number of variations and modifications can be made without departing from the scope of the invention as defined in the claims.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

The examples are described for the purposes of illustration and are not intended to limit the scope of the invention.

### Example 1

### Preparations of Protective Agent Compositions

Polypodium extracts was reconstituted and diluted with solution.

In a round bottom flask of 50 ml. capacity, 10 mg of soluble collagen and 10 mg of elastin were combined. The mixture was solubilized in 10 ml_{—} of sterile saline solution (0.9%) with continuous stirring. In a separate 50 ml_{—} round bottom flask, 5 mg of sphingosine and 5 mg cerebroside were combined. This mixture was dissolved in pure ethanol. The alcohol was completely removed by rotary vacuum evaporation to obtain a uniform coating of the sphingosine and cerebroside on the flask wall. To this flask 400 units of Polypodium solution in 6 ml of diluting liquid was added. The flask was swirled and then stirred continuously for 5 minutes at room temperature to uniformly coat the Polypodium with the sphingosine and cerebroside micelle coating and suspend it in the solution. This coated, suspended and preserved micellar-like solution was then added to the flask containing the mixture of collagen and cross-linked, low molecular weight elastin. The solution was stirred for about 5 minutes and then kept at room temperature.

### Example 2

### Polypodium Cream Formulation

The stabilized Polypodium and carotenoid composition was formulated into a cream/lotion for topical administration as outlined below.

Total Volume of the cream/lotion (400 ml_)

### Phase A:

### De-ionized Water 74.7% Tetra Sodium EDTA 0.5-0.7% Methyl Paraben 0.2% Propylene

### Glycol 3.0%-4.0% Glycerin 3.0%-4.0%

### Phase B:

### Cetyl Alcohol (Ado 1 52 NE) 2.0%

### Cetearyl Alcohol 2.0%

### Glyceryl Stearate 2.0%

### PEG-100 Stearate 1-2%

### Stearic Acid (Emersol 132) 4.5%

### Sorbitan Palmitate 0.5-0.7%

### Polysorbate-85 1.0%

### Polysorbate 60 0.5-1%

### Lanolin Alcohol (Ritachol) 1.0%

### HoHoba Oil 0.5-1%

### Lanolin 1-2%

### Tocopheryl Acetate 0.5-1%

### Dimethicone 200 0.7-1.0%

### BHA 0.1%

### Propylparaben 0.1%

### Diazolidinyl UREA 0.2%

### Phase C:

Fragrance (lilac, jasmine) as needed
Aloe Vera (powder) 1.5%-2.0%

### CoQ-10 0.5%

### Retinyl A 0.03-0.05%

Hyaluronic Acid (pure) 1.0-1.5%

### Talcum Powder (TiO2) 1.0-1.5%

Phase D: d-limonene 0.7%

### Allantoin 0.5%

### Fulvic Acid 0.5%

Quillaja saponaria (QTS) 0.3%
Acanthophyllum squaimsom (ATS) 0.3%

### Myrrh Extract 0.2%

### Hydroquinone Glyquin 4.0% Phase E:

Stabilized Polypodium in Collagen Suspension Matrix 800 units

Procedure:

Heat Phase A and Phase B separately with agitation to 75 C. Add Phase A to Phase B and mix 30 minutes at 75 C. Cool down to 20-22 C and then add Phase C, D and E and continue to agitate until homogenous and one phase.

### Example 3

### Clinical Studies In Human Subjects and Use of Composition

### 10% Topical Polypodium Test Protocol

1. Use an 8-square photo-opaque cutout template with a cutout area of 1 cm². The cutouts will receive increasing increments of NB-UVB light (Dermalight-80, 311 nm UVB).
2. Apply a thin layer of topical polypodium to areas B and C on the midline and left lower back/left flank. Allow the polypodium to dry. Third subject had Vehicle to C.
3. Secure the template to area A (untreated area). Expose all cutouts for the first light dose. Then shield each cutout when it has received its target light dose. For example, skin type II will start with all cutouts exposed to 125 mJ/cm² NB-UVB light. Then cutout #1 of the template is shielded because it has received its target of 125 mJ/cm². The remaining cutouts are given the incremental increase of an additional 75 mJ/cm². Then cutout #2 is shielded after it received a cumulative dose of 200 mJ/cm². See table 1 for incremental increase based on Fitzpatrick skin type.
4. One hour after the application of the topical polypodium, repeat 4 and 5 to area B.
5. Three hours after the application of the topical polypodium, repeat 4 and 5 to area C.
6. Assess the areas 24 hours after the NB-UVB light exposure to find the MED.

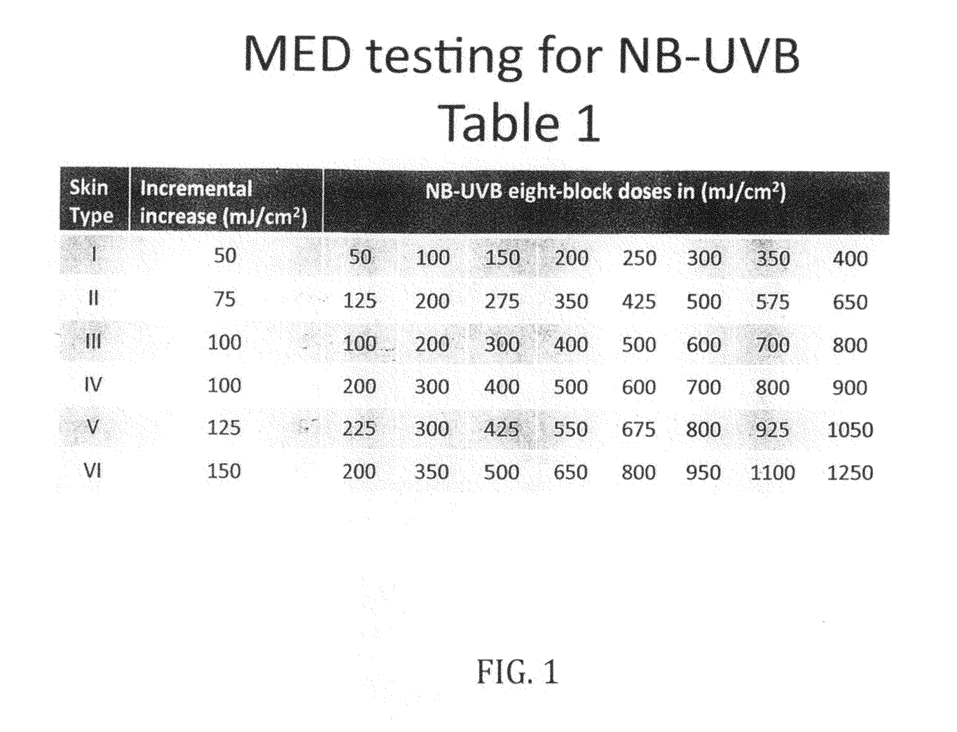

The effectiveness of the stabilized Polypodium was tested using a cream formulation. Human subjects applied the cream/lotion prior to exposure to UV light in accordance with the above protocol and testing schedule shown in FIG. 1. The subjects were photographed before and after the application of the cream on their backs and after the three time periods set forth.

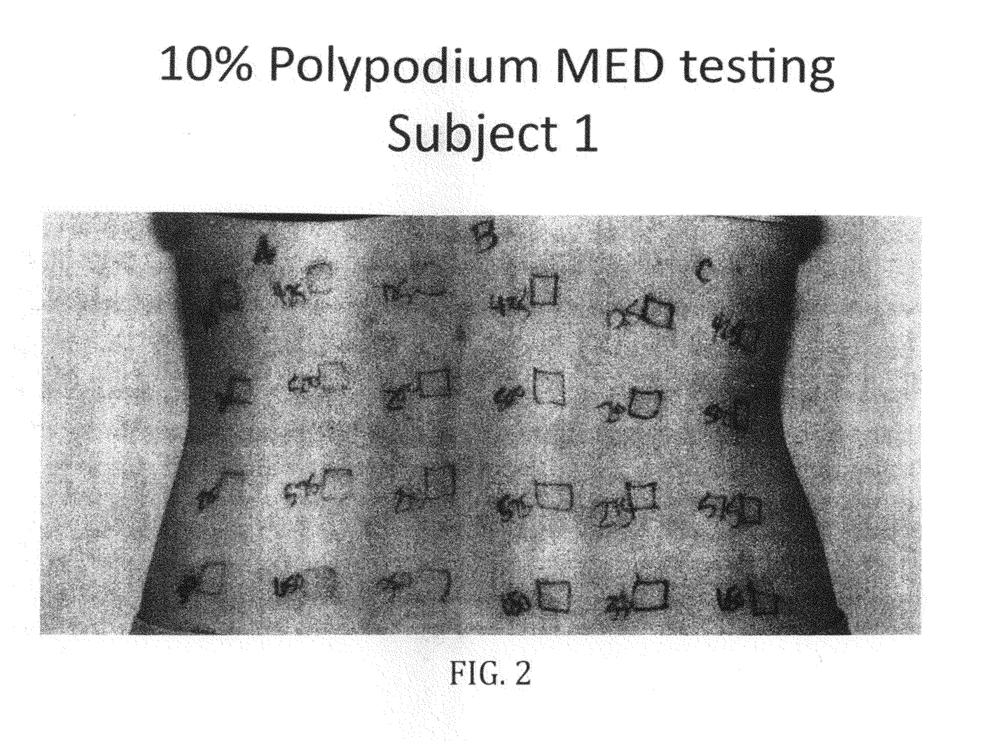

FIG. 2 is a photograph of the test area of Test Subject No. 1 with demarcations for test window identified thereon in relationship to a 10% polypodium MED test employing the composition of the present invention.

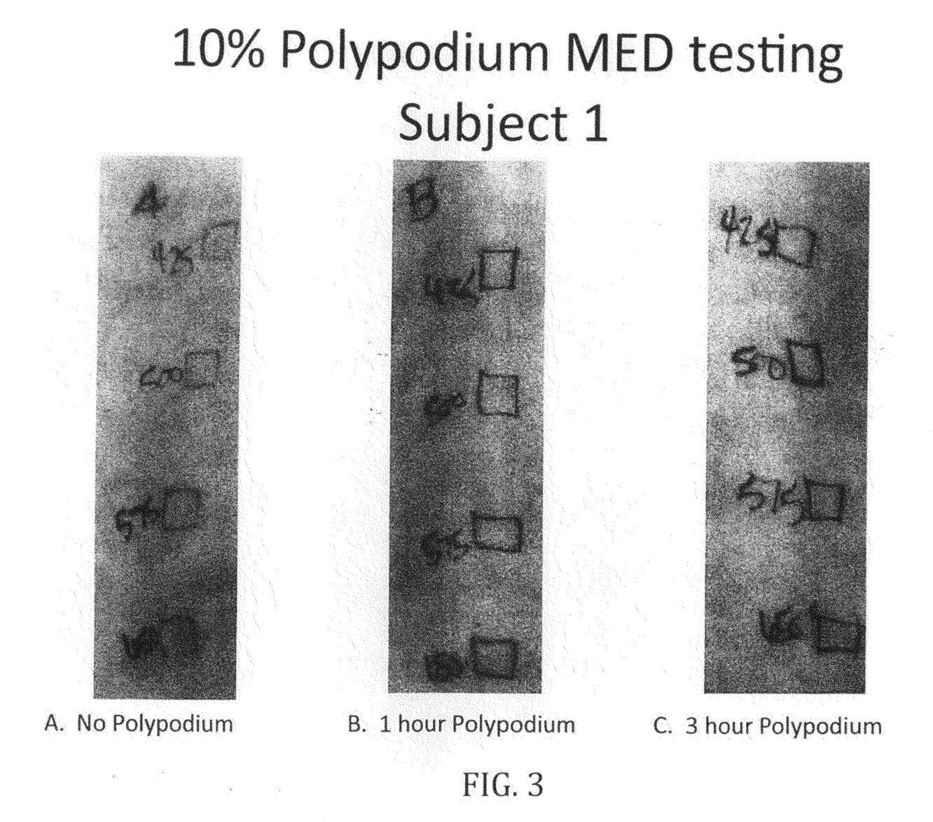

FIG. 3 is a series of photographs of Test Subject No. 1 with various levels of Polypodium and test protocol employing the composition of the present invention.

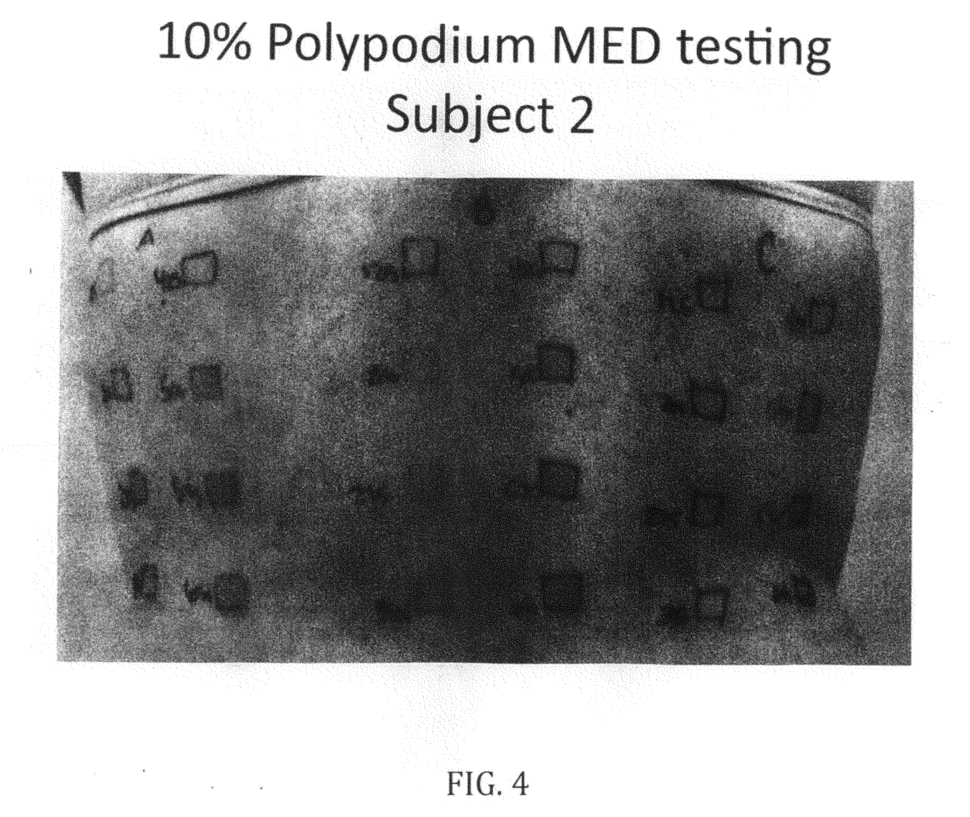

FIG. 4 is a photograph of the test area of Test Subject No. 2 with demarcations for test window identified thereon in relationship to a 10% polypodium MED test employing the composition of the present invention.

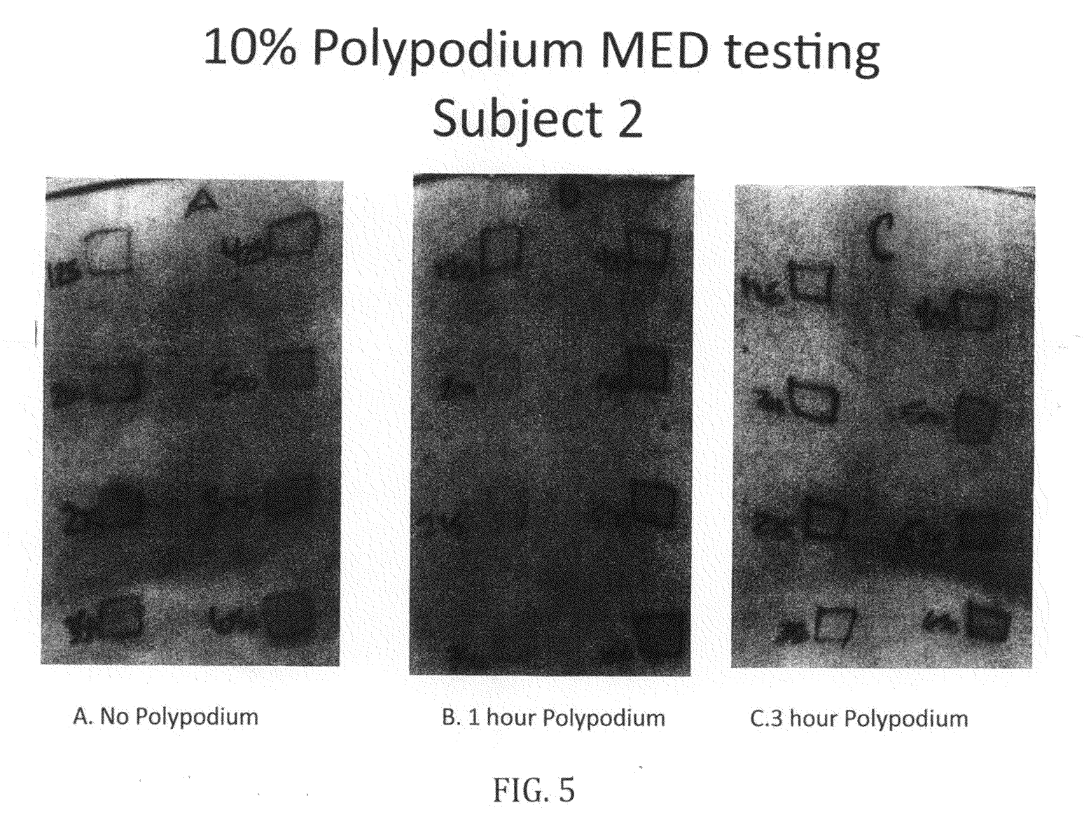

FIG. 5 is a series of photographs of Test Subject No. 2 with various levels of Polypodium and test protocol employing the composition of the present invention.

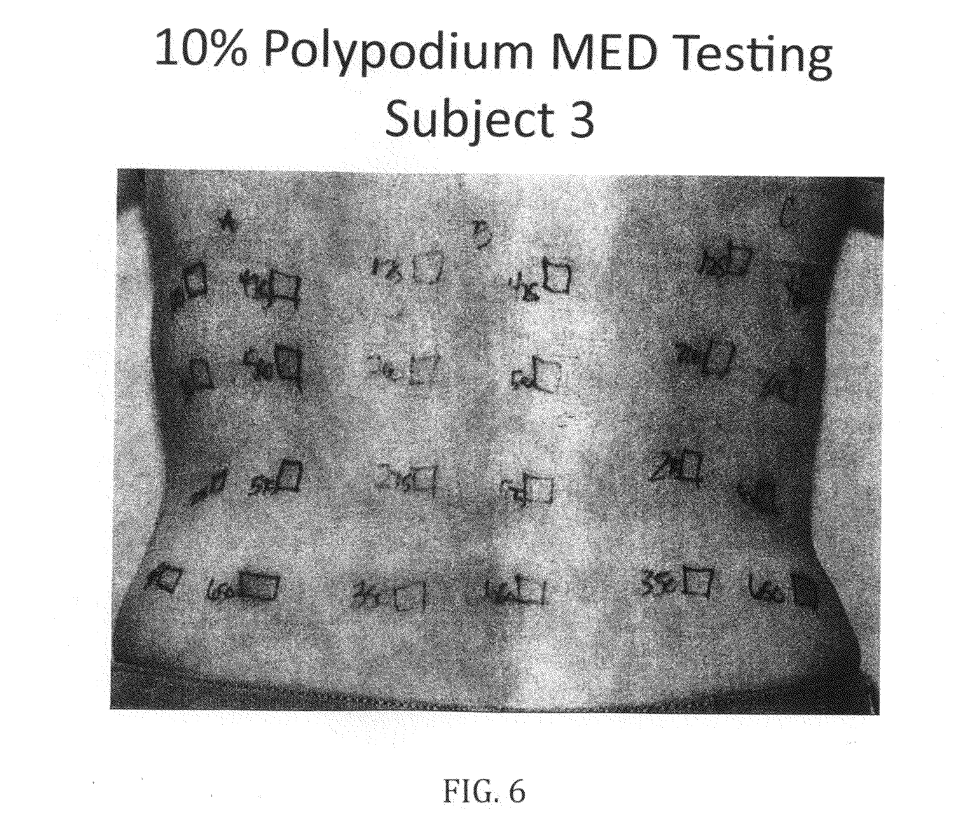

FIG. 6 is a photograph of the test area of Test Subject No. 3 with demarcations for test window identified thereon in relationship to a 10% polypodium MED test employing the composition of the present invention.

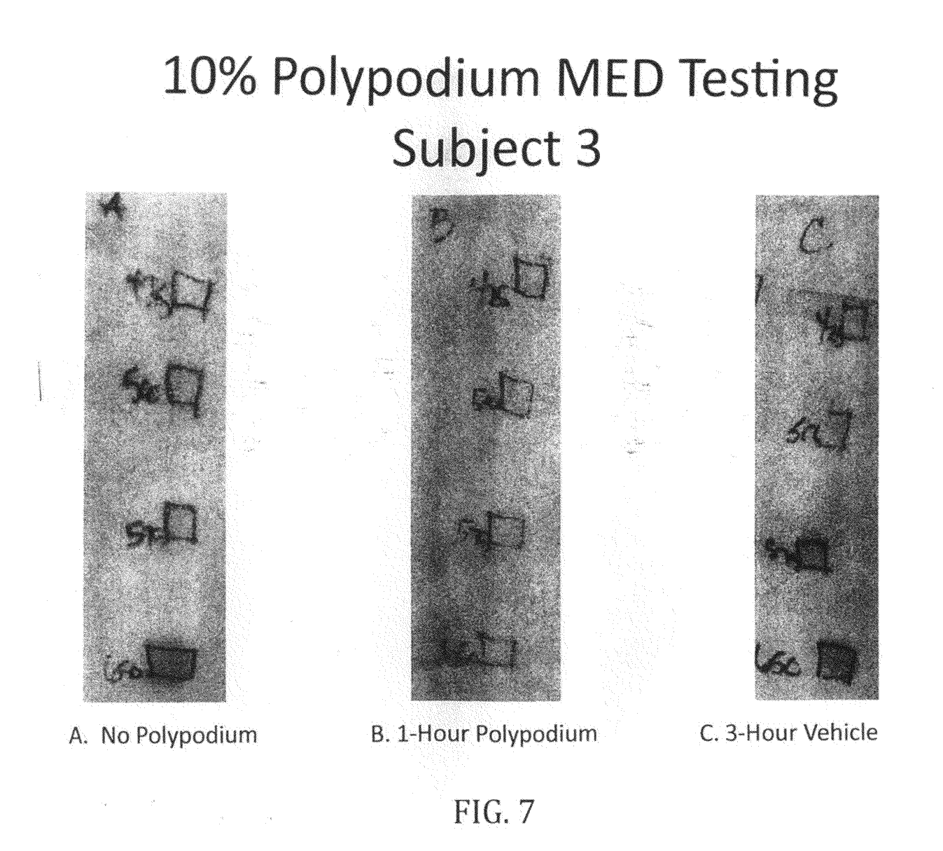

FIG. 7 is a series of photographs of Test Subject No. 3 with various levels of Polypodium and test protocol employing the composition of the present invention.

The foregoing description is meant to be illustrative and not limiting. Various changes, modifications, and additions may become apparent to the skilled artisan upon a perusal of this specification, and such are meant to be within the scope and spirit of the invention as defined by the claims.

What is claimed is:

1. A composition for topical delivery of at least one active agent, said composition comprising the active protective agent in a phospholipid micelle-like suspension and a pharmaceutically acceptable topical carrier.

2. The composition of claim 1 where the active protective agent is an effective amount of a Polypodium extract for photoprotection when applied topically to the skin and further comprising a composition selected from the group consisting of physical sunscreen agents, chemical sunscreen agents and cosmetic agents.

3. The composition of claim 1 wherein said carrier includes a chemical sunscreen agent.

4. The composition of claim 2 wherein said preparation includes at least about 1-50% Polypodium extract by weight.

5. The composition of claim 1 wherein said preparation provides a sun protection factor (SPF) for a minimal erythematic dose (MED) evaluated at 24 hours of at least 2 when applied at 2 μl/cm2 to normal skin of Type I to Type IV.

6. A sunscreen cosmetic having a sunscreen system comprising:
(i) from about 0.001 to about 20% by weight of green tea;
(ii) from about 0.001 to about 25% by weight of a active protective agent in a phospholipid micelle-like suspension which is effective to at least partially block ultraviolet radiation from harming human skin; and,
(iii) from about 30 to 99.9% by weight of a pharmaceutically acceptable carrier.

7. The composition according to claim 1 wherein the sunscreen compound is an organic material selected from the group comprising para-amino benzoates; salicylates; cinnamates; benzophenones; coumarins; azoles; menthyl anthralinates; polyethylene; polyamides and mixtures thereof.

8. The composition according to claim 1 wherein the sunscreen compound is inorganic and selected from the group comprising titanium dioxide, zinc oxide, iron oxide and combinations thereof.

9. The composition of claim 1 further comprising least one carotenoid selected from the group comprising astaxanthin, canthaxanthin, β-carotene, lutein, and zeaxanthin.

10. The composition of claim 9 wherein said composition provides a sun protection factor of at least 5-15.

11. The composition according to claim 2 wherein said extract is a Polypodium aureum extract.

12. The composition according to claim 2 wherein said extract is a Polypodium crassifolium extract.

13. The composition according to claim 2 wherein said extract is a Polypodium decumanum extract.

14. The composition according to claim 2 wherein said extract is a Polypodium lanceolatum extract.

15. The composition according to claim 2 wherein said extract is a Polypodium leucotomos extract.

16. The composition according to claim 2 wherein said extract is a Polypodium percussum extract.

17. The composition according to claim 2 wherein said extract is a Polypodium triseriale extract.

18. The composition according to claim 2 wherein said extract is a Polypodium vulgare extract.

19. The composition according to claim 1 wherein the carrier includes a physical sunscreen agent selected from the group consisting of titanium dioxide, silicone-treated titanium dioxide, zinc oxide, talc.

20. The composition according to claim 2 further comprising collagen.

21. The composition according to claim 20 further comprising elastin.

22. A method for making a stabilized active protective agent composition, said method comprising the steps of:
i. dissolving a phospholipid in a solvent to prepare a phospholipid solution; and
ii. admixing the phospholipid solution with an active protective agent so as provide a phospholipid micelle-like encapsulated active protective agent suspension.

23. A method according to claim 22, further comprising the step of combining the micelle-like suspension with a solution of solubilized collagen and elastin.

24. The method of claim 23, wherein the phospholipid is selected from the group consisting of shingosine, cerebroside and combinations thereof.

25. A method of preparing a pharmaceutical or cosmetic formulation for delivery of an active protective agent, said method comprising the steps of preparing a composition as defined in claim 1 and combining the composition with additional ingredients to form a cream, lotion, gel, ointment, or spray.

26. A method of assisting in the reconstruction of photaged and damaged skin and preventing further photoaging comprising applying a composition according to claim 1.
